(19) United States
(12) Patent Application Publication
(10) Pub. No.: US 2016/0366886 A1
(43) Pub. Date: Dec. 22, 2016

(54) BIOCIDAL COMPOSITION, PREPARATION AND METHODS OF USE THEREOF

(71) Applicant: BROMINE COMPOUNDS LTD., Beer Sheba (IL)

(72) Inventors: SHLOMO ANTEBI, HAIFA (IL); DAVID FELDMAN, HAIFA (IL)

(21) Appl. No.: 15/121,073

(22) PCT Filed: Feb. 26, 2015
(86) PCT No.: PCT/IL2015/050218

§ 371 Date: Aug. 24, 2016

Related U.S. Application Data
(60) Provisional application No. 61/945,169, filed on Feb. 27, 2014.

(51) Int. Cl. A01N 43/64; A61L 2/18; A61L 2/23; A01N 59/00

(52) U.S. Cl. CPC A01N 43/64; A01N 59/00; A61L 2/18; A61L 2/23

(57) ABSTRACT

This invention is directed to a biocidal, antifouling or disinfection composition and methods of use thereof for inhibiting growth of microorganism and biofilms, reducing the microbial activity and killing microorganism in an aqueous system or on solid surfaces, wherein the biocidal composition includes stabilized chlorine compound such as dichloroisocyanurate salt, ammonium salt and a base.

### FIELD OF THE INVENTION

This invention is directed to a biocidal, disinfection and antifouling composition and methods of use thereof for inhibiting growth of microorganism and biofilmreducing the microbial activity and killing microorganism in an aqueous system or on solid surfaces, wherein the composition includes stabilized chlorine compound such as dichloroisocyanurate salt, ammonium salt and a base.

### BACKGROUND OF THE INVENTION

Industrial process water, circulating water, water supply lines and surfaces which are in contact with water are often observed with microorganism growth (such as algae, fungi, bacterial and others) on their surfaces in contact with water. Such microorganism growth may cause health problems and infectious diseases, impair the quality of products, disturb production processes, reduce the effectiveness of a pipe or conduit, causes corrosion of pipes and chests, causes problems in paper or board machines, increases deterioration of the quality of finished paper and cause foul odors and the like.

Haloamines are known biocides for reducing, inhibiting and/or controlling the proliferation of such microorganism growth.

Mixture of a solution of an ammonium salt and a solution of hypohalite (hypochlorite: Off) at elevated pH is known to produce a haloamine biocidal agent in situ; and has been reported as an effective biocide. The resultant is not stable and decomposes quickly followed by drop of pH.

Furthermore, sodium hypohalite is not stable and degrades quickly over time.

Accordingly, there is a need for safer and stable starting materials to produce the biocide and methods for the preparation thereof.

### SUMMARY OF THE INVENTION

In one embodiment, this invention is directed to a biocidal, disinfection and antifouling composition comprising stabilized chlorine compound, ammonium salt and a base.

In another embodiment, the stabilized chlorine compound is TCCA, dichloroisocyanurate, dichlorodimethyl hydantoin, chlorobromodimethyl-hydantoin, salt thereof or combination thereof.

In one embodiment, this invention is directed to a biocidal, disinfection and antifouling composition comprising dichloroisocyanurate salt, ammonium salt and a base.

In one embodiment, this invention is directed to a method of inhibiting and preventing growth of microorganism and biofilm, reducing the microbial activity and killing microorganism in an aqueous system or on solid surfaces comprising treating the aqueous system or the solid surface with an effective amount of a biocidal, antifouling and disinfection composition comprising stabilized chlorine compound, ammonium salt and a base.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts biocidal efficacy of AmBrNaDCC:Na₂CO₃, 1.2:1:1.5 mol, towards Alginate beads; pH 7 of the treated water (Example 9) at different concentrations.

FIG. 2 depicts biocidal efficacy of activated AmBr, towards Alginate beads, pH 7 at different concentrations.

FIG. 3 depicts biocidal efficacy of chloramine prepared by mixture of ((NH₄)₂SO₄:NaOCl 1:2 mol), towards Alginate Beads, pH 7 at different concentrations.

FIG. 4 depicts biocidal efficacy of NaOCl towards Alginate beads, pH=7 at different concentrations.

FIG. 5 depicts biocidal efficacy of NH₄Cl:NaDCC:NaOH 1.2:1:2.01 mol, towards Alginate beads, pH 7 at different concentrations.

FIG. 6 depicts biocidal efficacy of trichloroisocyanuric acid (TCCA) solution towards Alginate Beads, pH 7 at different concentrations.

FIG. 7 depicts biocidal efficacy of sodium dichloroisocyanurate (NaDCC) towards Alginate beads, pH 7 at different concentrations.

FIG. 8 depicts biocidal efficacy of several biocides at 0.5 ppm dose.

FIG. 9 depicts biocidal efficacy of several biocides at 1 ppm dose.

FIG. 10 depicts biocidal efficacy of several biocides at 2.5 ppm dose.

FIG. 11 depicts biocidal efficacy of several biocides at 5 ppm dose.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one embodiment, this invention is directed to a biocidal, antifouling and disinfection composition comprising an oxidant, ammonium salt and a base. In another embodiment, the oxidant is an active halogen. In another embodiment, the oxidant is a chlorine precursor. In another embodiment, the oxidant is a stabilized chlorine compound. In another embodiment, this invention is directed to a biocidal, disinfection and antifouling composition comprising stabilized chlorine compound, ammonium salt and a base. In another embodiment, the stabilized chlorine compound is TCCA, dichloroisocyanurate salt, dichlorodimethyl hydantoin, chlorobromodimethyl hydantoin or combination thereof. In another embodiment, the stabilized chlorine compound is dichloroisocyanurate salt, trichloroisocyanuric acid (TCCA) or combination thereof. In another embodiment, the dichloroisocyanurate salt is sodium dichloroisocyanurate (NaDCC).

The term “stabilized chlorine compound” refers to a compound that releases active chlorine upon dissolution in water.

The concentration of the active chlorine is based on the measurement of total chlorine (as Cl₂). The term “as Cl₂” refers to the measurement of the active chlorine as total chlorine.

In one embodiment, the concentration of the active chlorine in the composition of this invention, wherein the composition is an aqueous solution, is between 0.5 to 2000 ppm. In another embodiment, the concentration of the active chlorine is between 0.5 to 1000 ppm. In another embodiment, the concentration of the active chlorine is between 0.5 to 500 ppm. In another embodiment, the concentration of the active chlorine is between 0.5 to 100 ppm. In another embodiment, the concentration of the active chlorine is between 10 to 2000 ppm. In another embodiment, the concentration of the active chlorine is between 10 to 1000 ppm. In another embodiment, the concentration of the active chlorine is between 10 to 500 ppm. In another embodiment, the concentration of the active chlorine is between 10 to 100 ppm. In another embodiment, the concentration of the active chlorine is between 500 ppm to 2000 ppm. In another embodiment, the concentration of the active chlorine is between 500 ppm to 1000 ppm. In another embodiment, the concentration of the active chlorine is between 1000 ppm to 1200 ppm. In another embodiment, the concentration of the active chlorine is between 1000 ppm to 1500 ppm. In another embodiment, the concentration of the active chlorine is between 1500 ppm to 2000 ppm. In another embodiment, the concentration of the active chlorine is between 0.5 ppm to 10 ppm, when a solid composition is added directly to the aqueous system.

The term “ppm” refers to a concentration of one milligram material in one liter solution.

In another embodiment, the biocidal, antifouling and disinfection composition of this invention and methods of use thereof comprise and make use of dichloroisocyanurate salt, ammonium salt and a base. In another embodiment, the molar ratio between the dichloroisocyanurate salt (as Cl₂), ammonium salt and the base is 1:1.2:1.5, respectively. In another embodiment, the molar ratio between the dichloroisocyanurate salt (as Cl₂) and the base is between 1:1.5 to 1:6, respectively. In another embodiment, the molar ratio between the dichloroisocyanurate salt (as Cl₂) and the base is between 1:1.5 to 1:2, respectively. In another embodiment, the molar ratio between the dichloroisocyanurate salt (as Cl₂) and the base is 1:1.5 respectively. In another embodiment, the molar ratio between the dichloroisocyanurate salt (as Cl₂) and the base is 1:2 respectively. In another embodiment, the molar ratio between the dichloroisocyanurate salt (as Cl₂) and the base is 1:3 respectively. In another embodiment, the molar ratio between the dichloroisocyanurate salt (as Cl₂) and the base is 1:4 respectively. In another embodiment, the molar ratio between the dichloroisocyanurate salt (as Cl₂) and the base is 1:5 respectively. In another embodiment, the molar ratio between the dichloroisocyanurate salt (as Cl₂) and the base is 1:6 respectively. In another embodiment, the composition is an aqueous solution. In another embodiment, the composition is a solid form.

In another embodiment, the molar ratio between the ammonium salt:dichloroisocyanurate salt (as Cl₂): base in the biocidal, antifouling and disinfection composition is about 0.93:1:1.25 (when the dichloroisocyanurate salt is in its hydrated form, calculated as Cl₂) or 1.08:1:1.45 when the NaDCC is dry without the hydrate, wherein the composition is a solid composition. In another embodiment, the molar ratio between the ammonium salt:dichloroisocyanurate salt (as Cl₂):base in the biocidal, antifouling and disinfection composition is about 1:1:1.25 (when the dichloroisocyanurate salt is in its hydrated form) or 1:1:1.45 when the NaDCC is dry without the hydrate, wherein the composition is a solid composition. In another embodiment, the molar ratio between the ammonium salt:dichloroisocyanurate salt (as Cl₂):base in the biocidal, antifouling and disinfection composition is between about 1:1:1 to 1:1:3. In another embodiment, the molar ratio between the ammonium salt:dichloroisocyanurate salt (as Cl₂):base in the composition of this invention is 1:1:2. In another embodiment, the molar ratio between the ammonium salt:dichloroisocyanurate salt (as Cl₂):base in the composition of this invention is 1:1:3.

In another embodiment, the biocidal, antifouling and disinfection composition is a solid composition/form. Non limiting examples of a solid composition comprising the antifouling and disinfection composition of this invention include a tablet, a capsule, a briquette, a granule or a pellet. In one embodiment, the solid composition is a pellet. In one embodiment, the solid composition is a briquette. In one embodiment, the solid composition is a granule. In one embodiment, the solid composition is a capsule. In one embodiment, the solid composition is a tablet.

In another embodiment, the tablet is an effervescent tablet. In another embodiment, the solid composition further includes a solid diluent, binder anticaking agent or combination thereof. In another embodiment, an effervescent tablet composition includes stabilized chlorine compound, a base, adipic acid or citric acid and an ammonium salt. In another embodiment, the stabilized chlorine compound is dichloroisocyanurate salt. In another embodiment, the base is sodium bicarbonate, sodium carbonate or combination thereof. In another embodiment, the ammonium salt is ammonium bromide.

In another embodiment, an effervescent tablet is prepared by mixing AmBr, dichloroisocyanurate salt and NaHCO₃ or Na₂CO₃ with either adipic acid or citric acid. In another embodiment, an effervescent tablet is prepared by mixing AmBr, dichloroisocyanurate salt (such as Na-DCC) and NaHCO₃ or Na₂CO₃ (portion A) with either adipic acid or citric acid and with either NaHCO₃ or Na₂CO₃ (portion B). The weight ratio between portion A and portion B (A:B) may change from 9:1, 8:2 and 7:3 up to 1:1 and less. The best ratio to provide good disintegration process and an appropriate final pH (above 9.5) upon dissolution is preferred.

In another embodiment, the tablet is added to a dissolution apparatus where the tablets are put in the dissolution apparatus and water is passed through to obtain a low concentration of the biocide (ppm level) for treating the water.

In one embodiment, the solid composition includes two layers, wherein the first layer comprises ammonium salt and the second layer comprises dichloroisocyanurate salt and a base. In another embodiment, the base is sodium carbonate or sodium bicarbonate or combination thereof.

In one embodiment, the solid composition includes three layers, wherein the first layer comprises ammonium salt, the middle layer comprises a base, and the third layer comprises dichloroisocyanurate salt. In one embodiment, the base prevents any contact between the ammonium salt and the dichloroisocyanurate salt. In another embodiment, the base is sodium carbonate or sodium bicarbonate.

In one embodiment, the biocidal, antifouling and disinfection composition and methods of use thereof comprise and make use of an ammonium salt. In another embodiment, the ammonium salt is ammonium bromide, ammonium chloride, ammonium sulfate, ammonium acetate, ammonium phosphate, ammonium formate, ammonium thiocyanate, ammonium hydroxide, ammonium carbamate or combination thereof. In another embodiment, the ammonium salt is a polymer ammonium salt as described in WO 2008/083263 which is incorporated herein by reference. In another embodiment, the ammonium salt is ammonium bromide. In another embodiment, the ammonium salt is ammonium chloride. In another embodiment the ammonium salt is ammonium sulfate.

In one embodiment, the biocidal, antifouling and disinfection composition and methods of use thereof comprise and make use of a base. In another embodiment, the base is NaOH, KOH, Na₂CO₃, NaHCO₃ or any combination thereof. In another embodiment, the pH of the biocidal, antifouling and disinfection composition, wherein the composition is an aqueous solution is a pH above 8.5. In another embodiment, the concentration of the base in the composition is a function of the concentration of the ammonium salt. In another embodiment, the concentration of the base is about 1.5 times (molar ratio) higher than the ammonium salt, wherein the composition is an aqueous solution. In another embodiment, the concentration of the base is about 1.1 to 5 times (molar ratio) higher than the ammonium salt, wherein the composition is an aqueous solution or in a solid form. In another embodiment, the concentration of the base is between 500 to 5000 ppm In another embodiment, the concentration of the base is between 500 to 1000 ppm. In another embodiment, the concentration of the base is between 1000 to 1500 ppm.

In another embodiment, the molar ratio between the ammonium salt:base in the biocidal, antifouling and disinfection composition is about 1: 1-5, respectively wherein the composition is a solid composition. In another embodiment, the molar ratio between the ammonium salt:base in the biocidal, antifouling and disinfection composition is about 1:1, respectively wherein the composition is a solid composition. In another embodiment, the molar ratio between the ammonium salt: base in the composition is about 1:2, respectively wherein the composition is a solid composition. In another embodiment, the molar ratio between the ammonium salt:base in the composition is about 1 :3, respectively wherein the composition is a solid composition. In another embodiment, the molar ratio between the ammonium salt:base in the composition is about 1:4, respectively wherein the composition is a solid composition. In another embodiment, the molar ratio between the ammonium salt:base in the composition is about 1:5, respectively wherein the composition is a solid composition. In another embodiment, the composition is an aqueous solution. In another embodiment, the composition is a solid form.

In another embodiment, the biocidal, antifouling and disinfection composition of this invention and methods of use thereof comprise and make use of dichloroisocyanurate salt, ammonium salt and a base. In another embodiment, the molar ratio between the dichloroisocyanurate salt (as Cl₂) and the ammonium salt is between 1:1 to 1:2 respectively. In another embodiment, the molar ratio between the dichloroisocyanurate salt (as Cl₂) and the ammonium salt is 1:1.2 respectively. In another embodiment, the molar ratio between the dichloroisocyanurate salt (as Cl₂) and the ammonium salt is 1:1.5 respectively. In another embodiment, the molar ratio between the dichloroisocyanurate salt (as Cl₂) and the ammonium salt is 1:1.7 respectively. In another embodiment, the molar ratio between the dichloroisocyanurate salt (as Cl₂) and the ammonium salt is 1:2 respectively.

In one embodiment, this invention is directed to a biocidal, antifouling and disinfection composition and methods of use thereof. In another embodiment, the composition is an aqueous solution. In another embodiment, the pH of the biocidal aqueous solution is between 8.5 to 11. In another embodiment, the pH of the biocidal aqueous solution is between 8.5 to 10. In another embodiment, the pH of the biocidal aqueous solution is 8.5. In another embodiment, the pH of the biocidal aqueous solution is 9. In another embodiment, the pH of the biocidal aqueous solution is 10 or 11.

In one embodiment, the biocidal, antifouling and disinfection composition as a solid form is stable for more than three months. In another embodiment, the biocidal, antifouling and disinfection composition as an aqueous solution is more stable than activated AmBr solution. In another embodiment, the materials used for the preparation of the composition of this invention are more stable compared to activated AmBr solution. In another embodiment, the biocidal composition is stable due to the cyanuric acid.

In one embodiment, the biocidal, antifouling and disinfection composition of this invention includes an active biocide with similar biocidal efficacy as activated AmBr or chloramine In another embodiment, the biocidal, antifouling and disinfection composition of this invention includes an active biocide with better stability than activated AmBr or chloramine

In some embodiments of the invention, the composition of this invention is formed and fed either batchwise or continuously by any suitable means, such as by a liquid metering pump or by gravity.

A continuously fed composition refers to a saturated composition (aqueous solution), or a solution having a required constant concentration which is continuously pumped/delivered with or without being diluted by water from a solution holding tank via a pump towards the point of use.

In one embodiment, the composition of this invention is prepared and used for treating water in aqueous systems or solid surface by mixing a solution comprising stabilized chlorine compound and a base with a solution comprising an ammonium salt or the addition of the two solutions is reversed. In another embodiment, the stabilized chlorine compound is Na-DCC.

In another embodiment, the volumes of the two solutions are equal. In another embodiment, the volumes of the two solutions is not equal, but keeping the recommended ratio between the components.

In one embodiment, the composition of this invention is prepared and used for treating water in aqueous systems or solid surface by mixing a solution comprising a stabilized chlorine compound and a base and the addition of this solution to an ammonium salt in its solid form. In another embodiment the solid form of the ammonium salt is a tablet or pellet.

In one embodiment, the composition of this invention is prepared and used for treating water in aqueous systems or solid surface by mixing Solution (A) including dichloroisocyanurate salt with Solution (B) including a base and adding Solution (C) including ammonium salt. In another embodiment, the solutions are added in parallel to the treated water, or by adding first Solution (A) and Solution (B) followed by the addition of Solution (C). The streams can be also added in parallel but to different sites of the pipeline and effluxed to the treated water.

In another embodiment, the molar ratio between dichloroisocyanurate salt (as Cl₂) and the base used for the preparation of the composition of this invention is between 1:1 to 1:6 respectively. In another embodiment, the molar ratio between dichloroisocyanurate salt (as Cl₂) and the ammonium salt used for the preparation of the composition of this invention is between 1:1 to 1:2 respectively.

In some embodiments of this invention, mixing a solution comprising stabilized chlorine compound and a base with a solution comprising an ammonium salt (or vise versa) is done continuously by the addition of concentrated solutions to a stream of water that dilutes the two streams to the required interacting concentration to obtain a diluted composition which is ready for use. In another embodiment, the concentration of the concentrated solutions is between 1000 to 4000 ppm each. In another embodiment, the concentration of the concentrated solutions is 1000 ppm each. In another embodiment, the concentration of the concentrated solution is 1500 ppm each. In another embodiment, the concentration of the concentrated solutions is 2000 ppm each. In another embodiment, the concentration of the concentrated solutions is 3000 ppm each. In another embodiment, the concentration of the concentrated solutions is 4000 ppm each. In another embodiment, when two equal volumes of concentrated solutions are interacted at 1000 ppm each (to result with an expected 500 ppm of active halogen. In another embodiment, the two solutions are at 2000 ppm each (to end with 1000 ppm of active halogen. In another embodiment, the two solutions are at 4000 ppm each. In accordance with other variations of this embodiment of the invention, the mixing takes place in a mixing chamber.

In another embodiment, a concentrated composition (0.5-2000 ppm of Cl₂) is prepared and diluted before usage. In one embodiment, the stability of the concentrated solutions and thereby concentrated composition is better than known activated AmBr biocide compositions. In another embodiment the concentrated composition (as an aqueous solution) is stable for between 1.5 to 3 hours.

In one embodiment, this invention is directed to a method of inhibiting growth of microorganism and biofilm, reducing the microbial activity and killing microorganism in an aqueous system or on solid surface in contact with an aqueous liquid comprising treating the aqueous system or the solid surfaces with an effective amount of the composition of this invention comprising stabilized chlorine compound, ammonium salt and a base. In another embodiment, the stabilized chlorine compound is a dichloroisocyanurate salt. In another embodiment, the stabilized chlorine compound is a sodium-dichloroisocyanurate. In another embodiment, the composition is optionally diluted prior to adding to the aqueous system.

In one embodiment, the term “treating” refers to applying. In another embodiment, the biocidal composition of this invention is applied to an aqueous system or to a solid surface which is in contact with an aqueous liquid.

In one embodiment, the term “effective” refers to the capability of the composition to inhibit growth of microorganism and biofilm, reducing the microbial activity and killing microorganism at least by 50% of the microorganism in the aqueous system or solid surface.

In one embodiment, this invention is directed to a method of inhibiting growth of microorganism and biofilm, reducing the microbial activity and killing microorganism in an aqueous system. In another embodiment, the method of this invention comprises adding to the aqueous system a dosage of between 0.1 ppm to 10 ppm of the composition of this invention. In another embodiment, the method of this invention comprises adding to the aqueous system a dosage of between 0.1 ppm to 1 ppm of the composition of this invention. In another embodiment, the method of this invention comprises adding to the aqueous system a dosage of between 1 ppm to 3 ppm of the composition of this invention. In another embodiment, the method of this invention comprises adding to the aqueous system a dosage of between 2 ppm to 5 ppm of the composition of this invention. In another embodiment, the method of this invention comprises adding to the aqueous system a dosage of between 2 ppm to 3 ppm of the composition of this invention. In another embodiment, the method of this invention comprises adding to the aqueous system a dosage of between 3 ppm to 4 ppm of the composition of this invention. In another embodiment, the method of this invention comprises adding to the aqueous system a dosage of between 3 ppm to 7 ppm of the composition of this invention. In another embodiment, the method of this invention comprises adding to the aqueous system a dosage of between 5 ppm to 10 ppm of the composition of this invention. In another embodiment, the biocidal composition is a solid composition. In another embodiment, the biocidal composition is an aqueous solution.

In one embodiment, the aqueous system being treated by the biocidal composition of this invention includes non limiting examples such as circulating water systems, swimming pools, cooling water system, circulating cool water, brewery pasteurizer, air washer, waste water, paper factory water, scrubbers, pond and lagoon water, surfaces, walls, floors, equipment, food plant disinfection, hospital care, meat disinfection and bleaching-pulp and paper.

In one embodiment, this invention is directed to a method of inhibiting growth of microorganism and biofilm, reducing the microbial activity and killing microorganism on solid surfaces. In another embodiment, the composition of this invention is an aqueous solution and is optionally diluted before applying to the solid surface to inhibit the growth of microorganism on the solid surface. In another embodiment, the composition is a solid composition and is dissolved in water to obtain a solution and thereby applying the solution on the solid surface to inhibit growth of microorganism on solid surface.

In one embodiment, the solid surfaces being treated by the composition of this invention include non limiting examples such as medical working space, medical devices, showers, bath, an interface between water and a surface of a solid in an industrial water environment.

In one embodiment, the method of this invention comprises treating aqueous systems or solid surfaces using the composition of this invention. In another embodiment, the composition of this invention is optionally diluted and applied immediately to the aqueous system or solid surface to be treated. In another embodiment, the biocidal composition is prepared prior to adding/applying to the aqueous system or the solid surface. In another embodiment, the composition is added batchwise or continuous.

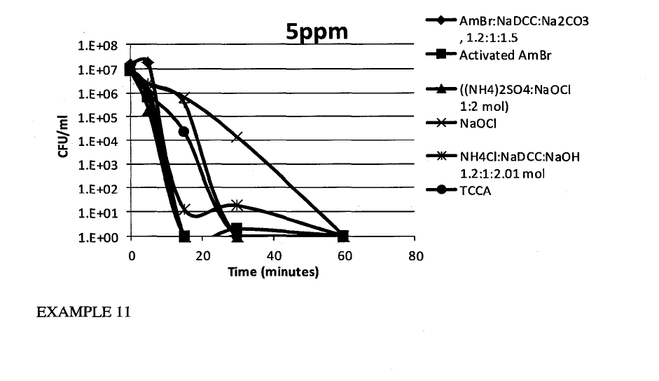

In one embodiment, the inhibiting growth of microorganism and biofilm, reducing the microbial activity and killing microorganism using the composition of this invention is effective as known haloamines as presented in FIGS. 10 and 11.

In another embodiment, this invention provides a method of inhibiting the microbial growth within 30 minutes after administration of 2.5 ppm of the biocidal composition of this invention by killing at least 90% of the microbe population. In another embodiment, this invention provides a method of inhibiting the microbial growth within 60 minutes after administration of 5 ppm of the biocidal composition of this invention by killing 100% of the microbe population.

In another embodiment, this invention provides a method of killing at least 90% of the microorganisms within 30 minutes after administration of 2.5 ppm of the biocidal composition of this invention. In another embodiment, this invention provides a method of killing 100% of the microorganism within 60 minutes after administration of 5 ppm of the biocidal composition of this invention.

In another embodiment, the biocidal composition is capable of reducing the microbial activity by at least 90% within 30 minutes after administration of 2.5 ppm of the biocidal composition of this invention. In another embodiment, the biocidal composition is capable of reducing the microbial activity by 100% within 60 minutes after administration of 5 ppm of the biocidal composition of this invention.

In another embodiment, the biocidal composition of this invention is an antifouling composition. In another embodiment, the methods of this invention are directed to control biofouling or preventing microbial fouling using the composition of this invention. In another embodiment, the fouling is due to algae, fungi, bacteria, and other living forms found in aqueous systems or solid surfaces.

The term “biocide” as used herein refers to a substance that kills microorganisms and their spores. Depending on the type of microorganism killed, a biocidal substance may be further defined as a bactericide (or antibacterial agent), a fungicide (or antifungal agent), algaecide. (anti-algae agent) a yeasticide (anti-yeast agent) etc.

The following examples are presented in order to more fully illustrate the preferred embodiments of the invention. They should in no way be construed, however, as limiting the broad scope of the invention.

### EXAMPLES

### Example 1

### Preparation of Activated AmBr

Activated AmBr solution was prepared by addition of NaOCl (1000 ppm) into an aqueous solution of ammonium bromide (1000 ppm, 1:1.2 molar ratio, respectively).

25 mL of ammonium bromide solution (166 mg (0.0169 M) in 100 ml) representing 1000 ppm of ammonium bromide (AmBr) was mixed with 25 mL of a solution prepared from 0.981 mL of 10.2% NaOCl (0.014 M) representing 1000 ppm as Cl₂. The resulting solution (pH dropped instantaneously from 10.53 to 9.8) was expected to yield 500 ppm of Cl₂ and presented UV absorption at 244 nm. The pH dropped gradually within 39 min from 9.8 to 8.03.

After 42 min a yellow solution was formed and the pH dropped to 3.29. The UV adsorption was still 244 nm and it increased slowly to 250 within 7 min (decomposition). The yellow solution is indicative to decomposition of the active species. With the addition of base no discoloration was observed.

UV absorption at 244 nm is characteristic of an active biocidal solution.

### Example 2

### Preparation of chloramine from NH₄Cl and NaOCl (1.2:1 molar ratio)

A chloramine solution was prepared by the addition of 100 ml of NaOCl (prepared from 981 μl of 10.2% NaOCl (0.014 M) representing 1000 ppm as Cl₂; to 100 ml aqueous solution of ammonium chloride (1000 ppm, prepared from 90.6 mg, NH₄Cl; Mw 53.49; 1.69 mmol in 100 ml H₂O,).

The resulting solution was highly basic with a pH 11.2, containing 539 ppm as Cl₂. UV absorption at 244 nm (characteristic absorption for chloramine) The solution was stable for 2 hrs, then a drop was observed. After 20.3 hrs the reading was 493 ppm (91%), after 44.4 hrs it dropped to 223 ppm (41.4%).

### Example 3

### Preparation of Haloamine Using AmBr and NaDCC.2H₂O (as Cl₂) (1.2:1 Molar Ratio)

100 mL of ammonium bromide (AmBr) solution (prepared from 166 mg AmBr (Mw 98) in 100 mL H₂O, 1.69 mmol, pH 5.62) was mixed with 100 mL of a solution prepared from 179 mg of NaDCC.2H₂O, Mw 255.98, 0.699 mole (1.4 mole. of Cl₂, pH 6.3). Seven minutes after the mixing a yellow solution was formed (pH 3.25, 256 nm, UV). The UV absorption was recorded at 261 nm after 51 min Monitoring the total active halogen (as Cl₂) showed a decreasing halogen level, i.e., 311 ppm (34 min) and 222 ppm (50 min) from 500 ppm (theoretical as Cl₂).

Due to low acidity (pH=6.3) the resultant haloamine was not stable and decomposed after 7 min.

UV absorption at 244 nm is characteristic of an active biocidal solution.

### Example 4

### Preparation of Haloamine (Composition of This Invention) Using AmBr, NaDCC.2H₂O (as Cl₂) and NaOH (1.2:1:1.5 Molar Ratio)

A solution of 100 mL of AmBr solution (1000 ppm; from 166 mg, 1.69 mmol in 100 ml H₂O, pH 5.58) was mixed with a solution of 100 mL of NaDCC.2H₂O (179 mg of NaDCC.2H₂O, Mw 255.98, 0.699 mole (1.398 mole of Cl₂, pH 6.3) and 0.57 g of a 15% aq. solution of NaOH (85.5 mg, 2.13 mmol, Mw 40), pH 12.1) resulting with a colorless solution having a pH 11.2, total active Cl₂ found 488 ppm (expected 500 ppm), UV 244 nm. The active Cl₂ level and UV remained unchanged after 3 hours.

The two solutions (AmBr (1) and (NaDCC+NaOH) (2)+were diluted to 1000 ppm of each reactant before mixing.

UV absorption at 244 nm is characteristic of an active biocidal solution.

### Example 5

### Preparation of Haloamine (Composition of This Invention) Using AmBr, NaDCC.2H₂O (as Cl₂) and NaOH in Different Ratios of NaOH

Experiments were conducted in order to optimize the molar ratio of NaOH required to obtain a stable solution:

### NH₄Br:NaDCC.2H₂O:NaOH 1.2:1:2 Molar Ratio

Solutions of AmBr and NaDCC.2H₂O were prepared as described in Example 3 and NaOH was added (by adding 0.755 g of a 15% aq. NaOH solution) to yield the desired molar ratio (NH₄Br:NaDCC.2H₂O:NaOH 1.2:1:2 molar ratio). The pH of the mixed solution increased to 11.67. This solution showed clearly a UV absorption at 244 nm, stable for an overnight period (18.5 hrs) keeping constant active Cl₂.

### NH₄Br:NaDCC.2H₂O:NaOH 1.2:1:1.1 Molar Ratio

Solutions of AmBr and NaDCC.2H₂O were prepared as described in Example 2 and NaOH was added (by adding 0.411 g of a 15% aq. NaOH solution) to yield the desired molar ratio (NH₄Br:NaDCC.2H₂O:NaOH 1.2:1:1.1 molar ratio). The pH of the mixed solution was 11.15. This solution showed a UV absorption at 244 nm (after 4 min) After 20 min the active Cl₂ level dropped to 20% of the starting level. The weight ratio between NaDCC.2H₂O:NaOH is 2.9.

UV absorption at 244 nm is characteristic of an active biocidal solution.

### Example 6

### Preparation of Haloamine (Composition of This Invention) Using AmBr, NaDCC.2H₂O (as Cl₂) and Na₂CO₃(1.2:1:1.5 Molar Ratio)

Experiments were conducted in order to optimize the molar ratio of Na₂CO₃ required to obtain a stable solution:

### NH₄Br:NaDCC.2H₂O:Na₂CO₃ 1.2:1:1.5 Molar Ratio

AmBr solution (100 ml, (from 166 mg, 1.69 mmol in 100 ml H₂O)), pH 5.58, was mixed with a solution of NaDCC.2H₂O (100 ml (from 179 mg of NaDCC.2H₂O, Mw 255.98, 0.699 mmol (1.398 eq. of Cl₂, pH 6.49) and of Na₂CO₃ (0.2245 g, Mw 106, 2.18 mmol), pH 10.54, resulting with a colorless solution having a pH 9.67. Total active Cl₂ found 489 ppm (expected 500 ppm), UV 243.8 nm The active Cl₂ level was stable for 77 min then started to decrease: loss of 10% after 3.25 hrs, 36% after 5.5 hrs then 45% after 5.55 hrs. This was accompanied with a pH change from 9.69 to 8.8.

The two solutions retained 1000 ppm of each reactant.

### NH₄Br:NaDCC.2H₂O:Na₂CO₃ 1.2:1:6 Molar Ratio

AmBr solution (100 ml, (from 166 mg, 1.69 mmol in 100 ml H₂O)), pH 5.58, was mixed with a solution of NaDCC.2H₂O (100 ml (from 179 mg of NaDCC.2H₂O, Mw 255.98, mole, 0.699 mole (1.398 eq. of Cl₂, pH 6.49) and of 0.9 g Na₂CO₃. the final pH of the solution after mixing was 10.73. This afforded a very stable solution in terms of the total chlorine level that did not change after 21 hrs.

The UV absorption was 244 nm after 4 min and 241.8 nm after 93 min However, no UV absorption at 244 nm was detected after 21 hrs, though the pH was still basic (pH 10.54). The active Cl₂ level remained almost unchanged after 21 hrs, similar to almost unchanged pH that dropped slightly from 10.74 to 10.54.

### NH₄Br:NaDCC.2H₂O:Na₂CO₃ 1.2:1:1 Molar Ratio

When the amount of Na₂CO₃ was decreased (to 1 mole) the UV absorption was observed at 242.8 nm after 6 min and disappeared (no peak was detected after 28 min) The total Cl₂ was already low after 2 min (loss of 11% of the expected Cl₂).

UV absorption at 244 nm is characteristic of an active biocidal solution.

### Example 7

### Preparation of Haloamine (Composition of this Invention) Using AmBr, Anhydrous NaDCC and Na₂CO₃ (1.2:1:3)

### NH₄Br:NaDCC:Na₂CO₃ 1.2:1:3 Molar Ratio

A solution of NaDCC (750 ml (from 1.224 g of NaDCC, Mw 219.95, 5.565 mmol (10.74 eq. of Cl₂) and of Na₂CO₃ (3.4 g, Mw 106, 32.08 mmol), pH 10.74 was added dropwise onto a tablet of AmBr (1.265 g, Mw 98, 12.91 mmol), resulting with a colorless solution having a pH 10.31. Total active Cl₂ found 806 ppm (expected 1000 ppm), UV 243 nm.

The active Cl₂ level was stable for 60 min then started to decrease: loss of 10% after 2 hrs, 20% after 3.4 hrs then 65% after 20 hrs. This was accompanied with a pH change from 10.31 to 9.92.

### Example 8

### Preparation of Haloamine Using (Composition of This Invention) AmCl, NaDCC.2H₂O (as Cl₂) and NaOH

### NH₄Cl:NaDCC.2H₂O:NaOH 1.2:1:2 Molar Ratio

Ammonium chloride (AmCl) solution (100 ml, (from 90.6 mg, 1.69 mmol in 100 ml H₂O)), pH 5.6, was mixed with a solution of NaDCC.2H₂O (100 ml (from 179 mg of NaDCC.2H₂O, Mw 255.98, 0.699 mole (1.398 eq. of Cl₂, pH 6.49) and of 15% aq. NaOH (0.755 g, Mw 40, 2 mmol), pH 10.19, resulting with a colorless solution having a pH 9.46. Total active Cl₂ found: 465 ppm (expected 500 ppm), UV 244 nm. The active Cl₂ level lost 10% after 5.5 hrs. After 3 days the active level was at 48% from starting level. This was accompanied with a pH change from 9.46 to 9.23.

UV absorption at 244 nm is characteristic of an active biocidal solution. In this Example chloramine was formed.

### Example 9

### Procedure for The Biocidal Tests

### Biocidal Activity Against Simulated Biofilm Systems

A biofilm simulation system developed by the Biofilm Bozeman Institute Montana (Grobe K. J., Zahler J., and Stewart P. S., 2002 in “Role of dose concentration in biocide efficacy against Pseudomonas aeruginosa Biofilms”, J. Industrial Microbiology & Biotechnology, vol. 29, pp 10-15), was used in this experiment to evaluate the efficacy of biocidal solutions against biofilm.

### Preparation of the Alginate Beads

The biofilm simulation was created by entrapping bacteria in alginate gel beads. A plate of R2A agar was streaked with Pseudomonas aeruginosa (ATCC 15442) and incubated at 35° C. overnight. Buffer phosphate at pH 7.2 was used to scrap off the bacteria from the agar plate and to create a suspension. The bacterial suspension was mixed with an equal volume of an aqueous 4% sodium alginate solution, to make a final 2% alginate solution. The alginate and bacterial slurry were placed in a 50 ml syringe attached to a 22 gauge needle, connected to a compressed air tank, allowing the syringe to be pressurized. At 20 psig pressure a stream of small drops was forced out and dropped into a stirred solution of 50 mM CaCl₂. The Ca⁺² cross linked the alginate, and semi solid beads with entrapped bacterial cells were formed. The beads were allowed to stir in the CaCl₂ solution for about 20 minutes, and then rinsed in a dilute 5 mM CaCl₂ solution. Several flasks containing 100 beads each were incubated overnight at 35° C. on a rotating shaker in a buffer solution (at pH 7) with 5 mM addition of CaCl₂ to maintain the beads structure. The resulting beads diameter is about 2 mm

### General Description of the Experiment

At the beginning of the experiment, the supernatant of the beads buffer suspension containing 5 mM CaCl₂ was decanted and replaced by the 100 ml biocide solution with the required concentration (between 0.5, 1, 2.5 and 5 ppm as total Cl₂).

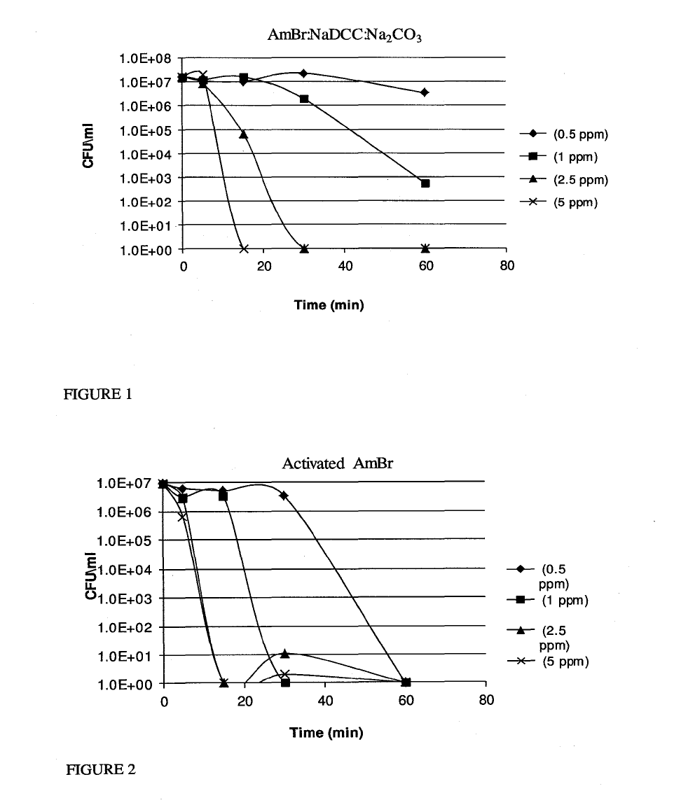

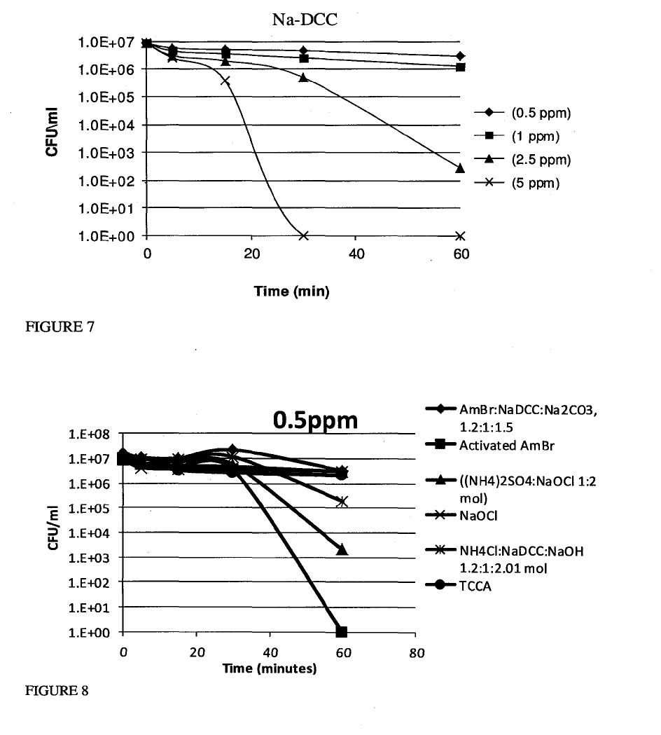

After different interval contact times, 10 beads were removed and placed in a 5 g/l sodium thiosulfate solution containing 50 mM sodium citrate. The sodium citrate was used to dissolve the alginate gel and release the bacteria into the solution. The neutralizer-citrate solution was placed in the refrigerator for 2 hours, than diluted and placed on R2A agar plates using pour plate technique. The plates were incubated at 35° C. for 24-48 hours and counted. The efficacy and toxicity of the neutralizer were checked as well as a control experiment without biocide addition. Four concentrations (0.5, 1, 2.5 and 5 ppm) were tested at four different contact times (5, 15, 30, and 60 min) FIGS. 1-10 describe the surviving colony forming units (CFU) of the bacteria after the biocide treatment at different contact times.

### Example 10

### Characterization of the Active Biocide of This Invention

Biocidal experiments differentiated between the different biocide solution. The efficacy of 4 different solutions was compared in the alginate beads test.

The biocide activity of AmBr:NaDCC:Na₂CO₃, 1.2:1:1.5 mol was compared to other biocides, i.e., activated AmBr, chloramine, trichloroisocyanuric acid (TCCA) and Na-DCC, using a synthetic biofilm-alginate beads.

### Biocide activity of AmBr/Na-DCC/Na₂CO₃ (Example 6)

The addition of 0.5 ppm dose did not have any effect on the microbe concentration. However, a 4 log reduction was achieved already with 1 ppm dose. A total kill was observed both for the two higher doses (2.5 and 5 ppm), differing in the time of kill, i.e. with a 5 ppm dose 15 min were sufficient to obtain a total kill, whereas with a 2.5 ppm dose 30 min were required (FIG. 1).

### Biocide Activity of Activated AmBr (Example 1)

In all doses (0.5 to 5 ppm) a total kill was observed, varying in the time of kill, i.e., 15 min for the 5 ppm dose to 30 min for 1 and 2.5 ppm, and 60 min for 0.5 ppm. (FIG. 2)

### Biocide Activity of Chloramine NH a Example 2)

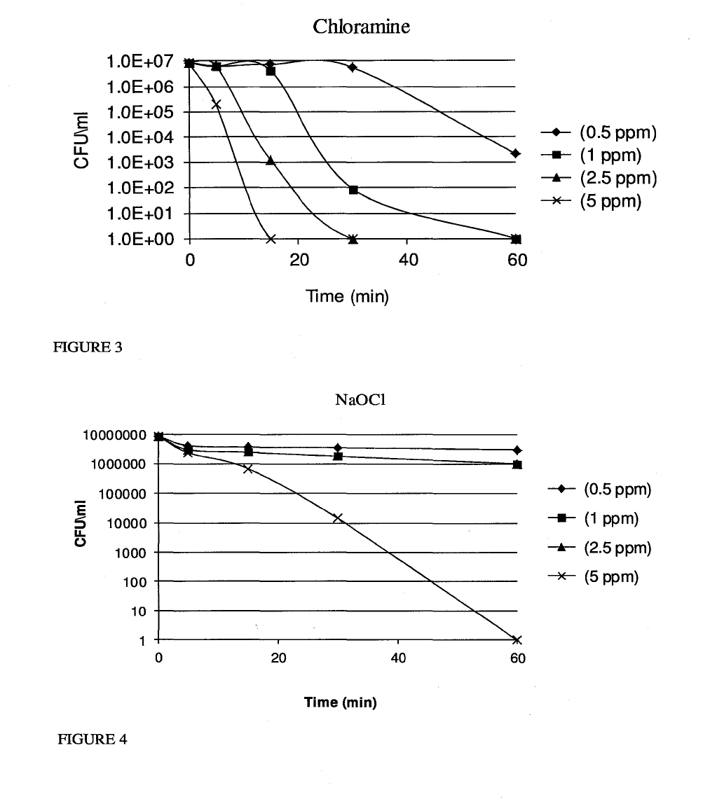

At the high doses, 2.5 and 5 ppm (FIG. 3), the behavior of the chloramine was similar to the solution of AmBr+NaDCC+Na₂CO₃ (FIG. 1). However, for the lower doses, 1 and 0.5 ppm, chloramine efficacy was better (FIG. 3) especially for the lower dose, 0.5 ppm, where a 4 log reduction was achieved whereas for the AmBr+NaDCC+Na₂CO₃ (FIG. 1) there was no effect at all. The performance of the chloramine is lower than that obtained with the activated AmBr

### Biocide Activity of NaOCl

The behavior of NaOCl towards alginated beads showed activity only at 5 ppm dose. At 5 ppm NaOCl a total kill was achieved, after 60 min., whereas for doses as low as 2.5 and 1 ppm only 1 log decrease was obtained and for 0.5 no effect was observed at all (FIG. 4).

### Biocide Activity of AmCl/NaDCC/NaOH

The biocide prepared from the mixture of AmCl/Na-DCC/Na₂CO₃ (ammonium chloride instead of ammonium bromide) lead unequivocally to the formation of chloramine.

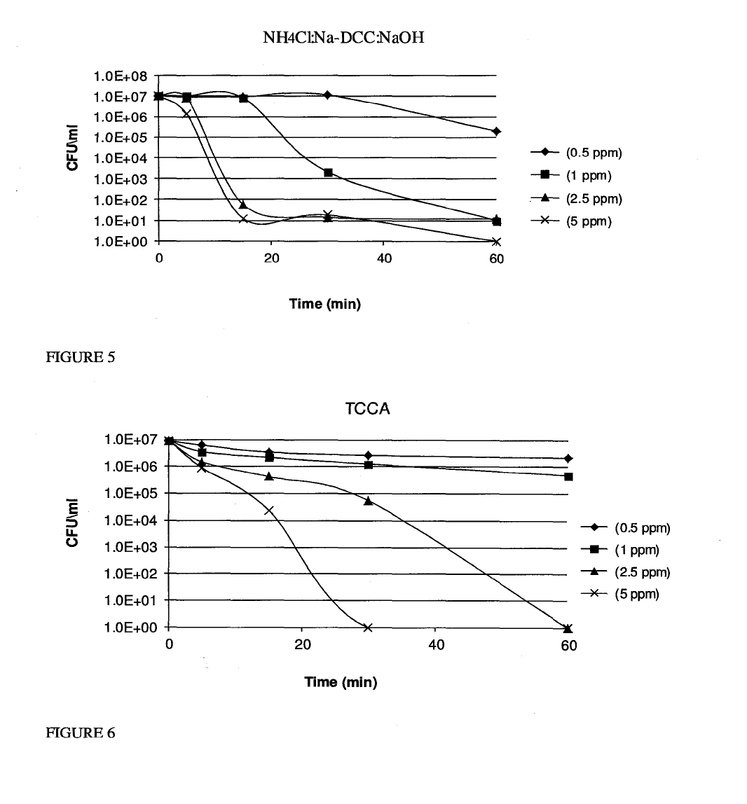

The behavior of AmCl/Na-DCC/Na₂CO₃ towards alginate beads showed activity similar to chloramine with the high doses of 2.5 and 5 ppm (FIG. 5). Total kill was achieved after 15 min. The 1 ppm dose gave a decrease of 4 log in the microbe concentration after 30 min., whereas the 0.5 ppm dose gave only 2 log reduction of the microbe concentration after 60 min A resemblance to the AmBr:NaDCC:Na₂CO₃ solution was noted.

### Biocide Activity of TCCA and NaDCC

The behavior of TCCA and NaDCC was similar (FIGS. 6 & 7), showing almost no activity with the low doses (0.5 and 1 ppm) and ending with a total kill for the 2.5 ppm dose after 60 min and for the 5 ppm dose after 30 min contact time. This suggested that the new AmBr:NaDCC:Na₂CO₃ is more active that the parent NaDCC.

### Differences Between Biocides Activity

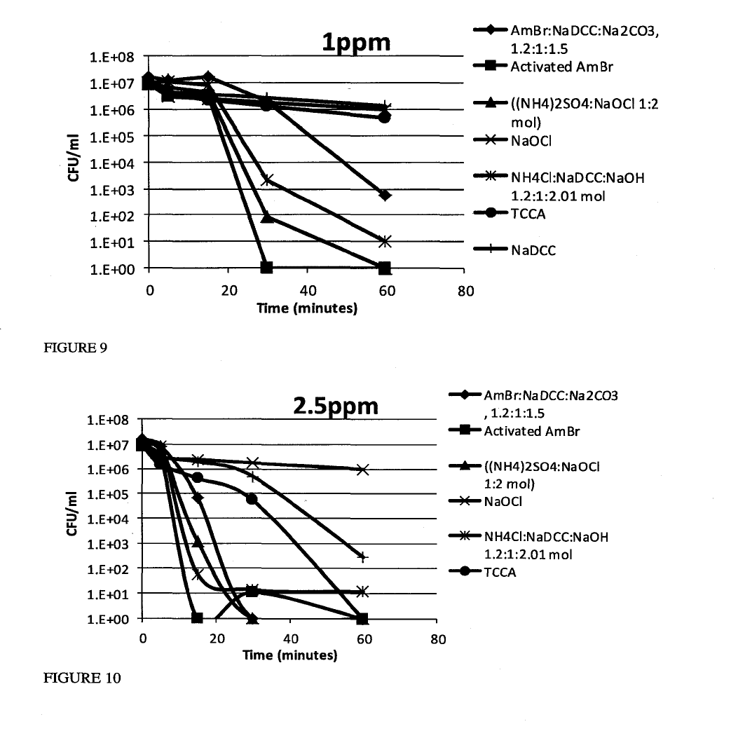

With a 0.5 ppm dose the biocidal efficacy of both activated AmBr and chloramine was the highest, favoring activated AmBr that achieved a total kill after 60 min. of contact time. Chloramine reduced the microorganism concentration by 4 logs (FIG. 8). At 1 ppm dose (FIG. 9) “formulations” NH₄Cl+NaDCC+NaOH, and NH₄Br+NaDCC+Na₂CO₃ demonstrated reduction of 6 log and 4 log, respectfully after 60 minutes. Activated AmBr demonstrated total kill after 30 min TCCA, NaDCC and NaOCl behaved similarly, almost with no biocidal efficiency (i.e. 1 log reduction).

At 2.5 ppm (FIG. 10) the biocidal behavior was almost the same for all the biocides excluding NaOCl, NaDCC and TCCA. After 60 min NaOCl remained inactive and showed only 1 log reduction of microbes kill TCCA achieved total kill after the same contact time, followed by NaDCC with more than 4 log reduction.

At 5 ppm dose total kill was achieved for all the biocides, insinuating that this is an overdose (FIG. 11).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A biocidal, disinfection and antifouling composition comprising stabilized chlorine compound, ammonium salt and a base.

2. The composition of claim 1, wherein the stabilized chlorine compound is dichloroisocyanurate salt, TCCA, dichlorodimethylhydantoin, chlorobromo dimethylhydantoin, or combination thereof; said ammonium salt is ammonium bromide, ammonium chloride, ammonium sulfate, ammonium acetate, ammonium formate, ammonium thiocyanate, ammonium hydroxide, ammonium carbamate or any combination thereof; said base is NaOH or Na₂CO₃; or any combination thereof.

3. (canceled)

4. The composition according claim 2, wherein said dichloroisocyanurate salt is sodium dichloroisocyanurate (Na-DCC).

5. (canceled)

6. (canceled)

7. (canceled)

8. The composition according to claim 2, wherein the molar ratio between said dichloroisocyanurate salt (as Cl₂) and said ammonium salt is between 1:1 and 1:2 respectively, or between 1:1.5 to 1:6 respectively.

9. (canceled)

10. The composition according to claim 1, wherein said composition is an aqueous solution.

11. The composition according to claim 10, wherein the pH of said aqueous solution is between 8.5-11.

12. The composition of claim 10, wherein the concentration of said active chlorine in the prepared biocidal solution is between 500-2000 ppm, or between 0.5-10 ppm.

13. (canceled)

14. The composition of claim 1, wherein said composition is a solid composition.

15. The composition of claim 14, wherein solid composition further comprises an acid.

16. The composition of claim 15, wherein said acid is adipic acid or citric acid.

17. The composition of claim 15, wherein said composition is prepared by mixing AmBr, dichloroisocyanurate salt and NaHCO₃ or Na₂CO₃ with either adipic acid or citric acid.

18. The composition of according to claim 14, wherein said solid composition is a tablet, a briquette, an effervescent tablet, granules or a pellet.

19. A method of inhibiting growth of microorganism and biofilm, reducing the microbial activity and killing microorganism in an aqueous system or on solid surfaces comprising treating the aqueous system or the solid surface with an effective amount of a biocidal, antifouling and disinfection composition comprising stabilized chlorine compound, ammonium salt and a base.

20. The method of claim 19, wherein said stabilized chlorine compound is dichloroisocyanurate salt, TCCA, dichlorodimethyl hydantoin, chlorobromo dimethyl hydantoin, or combination thereof.

21. The method of claim 19, wherein said composition is prepared by mixing a solution comprising stabilized chlorine compound and a base with a solution comprising an ammonium salt or the addition of the two solution is reversed, or wherein said composition is prepared by adding dropwise a solution comprising stabilized chlorine compound and a base onto a tablet, pellet or granules comprising an ammonium salt.

22. (canceled)

23. The method according to claim 19, wherein said composition is applied batchwise or continuously.

24. The method of claim 19, wherein said composition is added to said aqueous system to form an active chlorine level at a concentration of between 0.1 ppm to 10 ppm.

25. (canceled)

26. The method according to claim 19 wherein said stabilized chlorine compound is dichloroisocyanurate salt; said ammonium salt is ammonium bromide, ammonium chloride, ammonium sulfate, ammonium acetate, ammonium formate, ammonium thiocyanate, ammonium hydroxide, ammonium carbamate or combination thereof; said base is NaOH, NaHCO₃ Na₂CO₃ or combination thereof; or any combination thereof.

27. (canceled)

28. The method of claim 26, wherein the molar ratio between said dichloroisocyanurate salt and said ammonium salt is between 1:1 and 1:2 respectively, or between 1:1.5 to 1:6 respectively.

29. (canceled)

30. The method of claim 19, wherein said composition is an aqueous solution or a solid composition.

31. (canceled)

32. (canceled)

33. (canceled)
